(19) United States
(12) Patent Application Publication
(10) Pub. No.: US 2018/0177415 A1
(43) Pub. Date: Jun. 28, 2018

(54) CARDIOVASCULAR DISEASE DETECTION

(71) Applicant: Tamas Madl, Vienna (AT)

(72) Inventor: Tamas Madl, Vienna (AT)

(21) Appl. No.: 15/390,173

(22) Filed: Dec. 23, 2016

(51) Int. Cl. A61B 5/024; A61B 5/00; A61B 5/0456

(52) U.S. Cl. CPC A61B 5/02405; A61B 5/7275; A61B 5/746; A61B 5/7235; A61B 5/0456

(57) ABSTRACT

Methods and systems for detecting a heart condition include measuring a user's heart beat information. A predictive model is applied that includes multiple individual predictors and a classifier. Each predictor maps the user's heart beat information to a respective value. The classifier indicates a likelihood of a heart condition based on the predictors and the user's heart beat information. An alert is issued if the likelihood is above a threshold value.

### BACKGROUND OF THE INVENTION

Heart rate variability (HRV) measures changes in heart rate over time, specifically in time series of intervals between heart beats (R-R intervals). HRV has been shown to provide information that predicts some types of heart disease, such as heart failure. Low HRV indicates reduced cardiac regulatory capacity and is a strong predictor of mortality and health problems.

However, existing HRV analyses are not reliable enough for widespread clinical practice. Indeed, no single existing predictor is powerful enough to provide good predictions on patients' conditions. Several families of predictors have been used, including statistical features, geometric features (based on empirical sample density distributions of R-R intervals), non-linear features (based on analytic techniques from non-linear dynamical systems to infer and characterize system behavior, including attractor reconstruction), and frequency domain processes (including the separation and analysis of spectral components at different frequencies.

### BRIEF SUMMARY OF THE INVENTION

A method for detecting a heart condition includes measuring a user's heart beat information. A predictive model is applied, using a processor, that includes multiple individual predictors and a classifier. Each predictor maps the user's heart beat information to a respective value. The classifier indicates a likelihood of a heart condition based on the predictors and the user's heart beat information. An alert is issued if the likelihood is above a threshold value.

A system for detecting a heart condition includes a sensor configured to measure a user's heart beat information. A disease detection module comprising a processor is configured to apply a predictive model that includes multiple individual predictors and a classifier. Each predictor maps the user's heart beat information to a respective value. The classifier indicates a likelihood of a heart condition based on the predictors and the user's heart beat information. An alert module is configured to issue an alert if the likelihood is above a threshold value.

### BRIEF DESCRIPTION OF THE DRAWINGS

FIG. 1 is a block/flow diagram of a method for detecting a heart condition in a user in accordance with the present principles;

FIG. 2 is a block diagram of a biological neuron-based artificial neural network in accordance with the present principles;

FIG. 3 is a block/flow diagram of a method for reconstructing and quantifying robust attractors in accordance with the present principles;

FIG. 4 is a block diagram of a user device for detecting a heart condition in a user in accordance with the present principles;

FIG. 5 is a block diagram of a model generation system for generating predictive models in accordance with the present principles; and

FIG. 6 is a block diagram of a processing system in accordance with the present principles.

### DETAILED DESCRIPTION

Embodiments of the present invention provide heartrate variability (HRV) systems and processes that recognize potentially deleterious conditions such that treatment can be administered. The present embodiments can thereby recognize existing cardiovascular diseases, diseases that impact the autonomic nervous system, and autonomic stress responses, as well as recognize the risk of future cardiovascular events, without the need for invasive tests or even the oversight of medical personnel.

To accomplish this, the present embodiments take measurements of a user's heart activity signal and record, e.g., fiducial points such as peaks in the signal by, for example, measuring optical signals, audio signals, microelectromechanical signals, or electrical signals using a device in the user's possession, such as a smartphone or simple home testing device. Using this recorded data, the present embodiments distinguish normal from pathological heart activity (using, e.g., machine learning techniques and a corpus of training data).

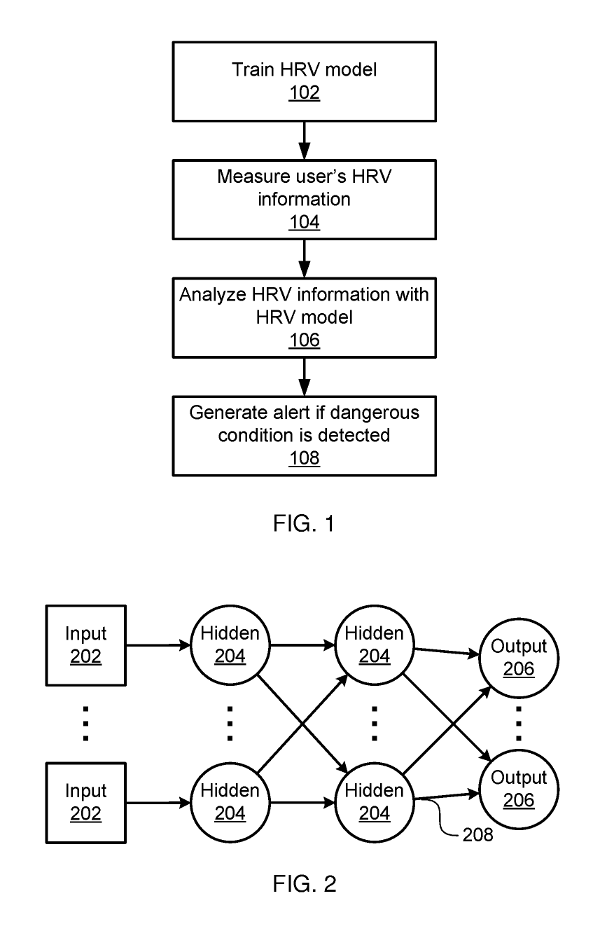

Referring now to FIG. 1, a method of detecting cardiovascular conditions is shown. Block 102 trains an HRV model using a corpus of HRV data. The HRV training data may include, e.g., measured heart beat information from a large group of people, converted to HRV information by measuring the time between heart beats (the R-R interval) and tracking how the R-R interval itself changes over time. The training data is used to train a model that is based on one or more disease predictors. Such predictors may include, e.g., statistical predictors, geometric predictors, neuron model-based predictors, neuronal equation learning predictors, attraction quantification predictors, graph-based predictors, discretization-based predictors, etc. Specific details on the types of predictors and how these predictors may be used and combined to form a model are provided below.

One a model for a given disease has been generated by block 102, block 104 measures heart beat information for individual users. These users may use, e.g., a device in their own possession such as, e.g., a smartphone or a dedicated medical sensing device to collect the heart beat information. Additional detail regarding the collection of heart beat information is provided below. The heart beat information is converted to HRV information. In an alternative embodiment, the measurement equipment may be located at a medical treatment facility.

Block 106 then uses the model provided by block 102 to determine whether the collected HRV information for the user is indicative of the disease or condition in question. For example, the model may provide a probability that the user has the disease or condition in question based on how well the measured HRV information matches the model. If the HRV information matches the model (e.g., if the probability exceeds a threshold value), block 108 generates an alert. This alert may be displayed to the user or, alternatively, to a medical care provider. In one embodiment, the alert module 108 may trigger an automatic treatment response, for example by triggering the automatic administration of medication.

In discussing the following predictors, the time series of R-R intervals of a given set of HRV data are designated as S=(t₁, t₂, . . . , tₙ), with each t representing a particular R-R interval.

Statistical Predictors

Statistical predictors of disease or other cardiovascular heart conditions include, e.g.:

Standard deviation: SDNN=σ(S)=\sqrt{Var(S)}.

Root mean squared standard deviation RMSSD=|S|⁻¹ \sqrt{Σᵢ₌₁^{|S|-1}(xᵢ₊₁−xᵢ)²}.

Ratio of the number of successive R-R interval differences that are greater than 20 ms to the total number of R-R intervals (pNN20=|xᵢ: (xᵢ₊₁−xᵢ)>20|/|S|).

Approximate entropy (ApEn(m,r,|S|)=Φ(r)−Φ² (r), calculated at four different values rϵ{0.1σ(S), 0.15σ(S), 0.2σ(S), 0.25σ(S)). Approximate entropy compares some number of consecutive values in the time series. Thus, m is the length of each run and r is a tolerance width. The logarithmic likelihood that runs of patterns that are close (within r) for contiguous observations remain close on subsequent incremental comparisons is measured. Φ is the averaged logarithm of the number of runs that are within this tolerance.

Geometric Predictors

Geometric predictors include, e.g.:

HRV triangular index:

$$HRI = \frac{\vert S \vert}{N} ,$$

where N is the total number of intervals in the modal bin of a histogram.

Spatial filling index

$$SFI = \frac{s}{{n}^{2}} ,$$

where s is a combined factor of the point distribution in phase space and n is the number of squares used to estimate the distribution.

Central tendency measure: CTM=Σₜ₌₁^{|S|-1}δ(Δᵢ), where δ(Δᵢ)=1 if and only if \sqrt{(xᵢ₊₂−xᵢ₊₁)²+(xᵢ₊₁−xᵢ)²}<r and zero otherwise.

Correlation dimension of a two-dimensional embedding.

Spectral power in particular frequency bands of a discrete Fourier transform.

Fluctuation exponents based on detrended fluctuation analysis. Detrended fluctuation analysis converts the entire time series into its profile, Xᵢ=Σₜ₌₁^{t}(xᵢ−<x>), and then divides the profile Xᵢ into time windows of length n and fits polynomials Yᵢ to each window using a least squares fit. The fluctuation function, a root mean square deviation from the trend is calculated: F(n)={square root over (N⁻¹Σₜ₌₁(Xₜ−Yₜ)̂2))}. This fluctuation function follows a power low, where F(n)∝n^{α} and the fitted exponent a may be used as an additional predictor.

The present embodiments make use of additional predictors beyond the above-described statistical and geometric predictors. In particular, multiple decorrelated predictors are used in the model, with more predictors resulting in higher predictive accuracy if they provide information that is not presented by the existing predictors. One additional predictor, which is inspired by knowledge about how heart beats are biologically controlled, is the biological neuron model-based predictor (BNM).

Biological Neuron-Based Predictor

The BNM predictor is based on the fact that mammalian heart beats are induced by a control mechanism that generates electrical impulses to precipitate muscular contraction. Impulses are generated by the sinoatrial node (SAN) and are propagated through the atrioventricular node (AVN) and His-Purkinje system, which are normally synchronizes with the activity of the SAN. The BNM provides an end-to-end differentiable neural network architecture that is based on non-linear coupling to simulated pacemaker neurons. Instead of tuning oscillator models to produce biologically plausible signals, an optimal ensemble of oscillators and an associated non-linear classifier are found, yielding the smallest misclassification error when classifying heart beat time series into healthy and pathological cases.

The BNM model accounts for cardiac activity and is based on the following differential equations:

$${\dot{v}}_{i} = {v}_{i} - \frac{{v}_{i}^{3}}{3} - {p}_{i , 1} {w}_{i} {v}_{i} + I$$

$${\dot{w}}_{i} = {p}_{i , 2} ( {v}_{i} - {p}_{i , 3} {w}_{i} )$$

where νᵢ corresponds to a membrane potential of a neuron i, wᵢ to the recovery variable of the neuron i, I is an external input, and p_{i,j} are model parameters, which govern the oscillatory dynamics. The dot operator indicates a derivative with respect to time.

A population of neurons is driven by a stream of action potentials which constitute the model input. This input is classified into healthy and pathological classes using a feed-forward neural network on top of the biologically inspired layer using the above equations to optimally fit and classify normal and pathological cardiac dynamics.

Referring now to FIG. 2, an exemplary artificial neural network is shown. An artificial neural network (ANN) is an information processing system that is inspired by biological nervous systems, such as the brain. The key element of ANNs is the structure of the information processing system, which includes a large number of highly interconnected processing elements (called “neurons”) working in parallel to solve specific problems. ANNs are furthermore trained in-use, with learning that involves adjustments to weights that exist between the neurons. An ANN is configured for a specific application, such as pattern recognition or data classification, through such a learning process.

ANNs demonstrate an ability to derive meaning from complicated or imprecise data and can be used to extract patterns and detect trends that are too complex to be detected by humans or other computer-based systems. The BNM-based ANN of FIG. 2 uses input neurons 202 that accept individual R-R intervals from a time series. Each input neuron 202 applies the above equations and outputs scalar firing rates to layers of hidden neurons 204, where the input neurons 202 “fire” when νᵢ exceeds a firing threshold parameterized by p_{i,4}:

$${f}_{i} = \{ \begin{matrix}{\sum }_{t = 0}^{T} \{ {v}_{i} ( t ) & if {v}_{i} ( t ) > {p}_{i , 4} \\ 0 & otherwise\end{matrix}$$

The input neuron firing rates fᵢ are fed into the neural network with hyperbolic tangent actication functions, multiple hidden layers, and a softmax output layer. Connections 208 between the input neurons 202 and hidden neurons 204 are weighted and these weighted inputs are then processed by the hidden neurons 204 according to some function in the hidden neurons 204, with weighted connections 208 between the layers. There may be any number of layers of hidden neurons 204, as well as neurons that perform different functions. Finally, a set of output neurons 206 accepts and processes weighted input from the last set of hidden neurons 204, providing a first output that reflects a likelihood that the time series represents a healthy patient and a second output that reflects a likelihood that the time series represents a patient with, e.g., ischemia or other disease or condition.

This represents a “feed-forward” computation, where information propagates from input neurons 202 to the output neurons 206. Upon completion of a feed-forward computation, the output is compared to a desired output available from training data. The error relative to the training data is then processed in “feed-back” computation, where the hidden neurons 204 receive information regarding the error propagating backward from the output neurons 206. Once the backward error propagation has been completed, weight updates are performed, with the weighted connections 208 being updated to account for the received error.

Because all numerical calculations in the BNM predictor are composed of a finite set of operations with known derivatives, the entire network is end-to-end differentiable. Reverse-mode automatic differentiation is used to train the network. More specifically, given known classes y, heart beat time series rr, and the parameter vector P, which includes neural network weights and biases as well as input neuron parameters, autograd is used to obtain a derivative of an L2 regularized, cross-entropy objective function:

$$J ( P ) = - \frac{λ}{n} {\vert \vert P \vert \vert}^{2} + \frac{1}{n} {\sum }_{k = 1}^{n} {y}_{k} log {O}_{I} ( {rr}_{k} , P ) + ( 1 - {y}_{k} ) log ( 1 - {O}_{I} ( {rr}_{k} , P ) )$$

where the first term represents L2 regularization, O₁(rrₖ, P) represents normalized probabilities of time series rrₖ containing indications for ischemia based on the described model, λ is a regularization parameter that reduces overfitting by helping enforce small values of P, and N is the number of data points in the training set. Once the gradient is obtained, a stochastic optimizer with momentum is used to find the optimal model parameters. The model is computationally expensive, so hyperparameters such as the number of layers, neurons per layer, minibatch size, and A may be adjusted by global black box optimization instead of a grid search to save computation time.

Initial weights for the connections 208 may be drawn from a normal distribution with σ=0.1 to break symmetry. The initial input neuron parameters p_{i,1-4} may be pre-optimized. To avoid the input neuron parameters adapting to the random initial weights 208, these parameters may be clamped in the first 10% of the training epochs. The weights 208 may then be allowed to change such that the rough initial parameters can be fine-tuned using gradient-based optimization.

Neuronal Equation Learning Predictor

Another predictor that may be employed is a neuronal equation learning predictor. In such a predictor, a neural network automatically learns the equations that best describe the input time series. To this end, instead of using one fixed type of activation function, neuron units are used with a large number of different functions including, but not limited to, sine, cosine, exponent, square root, and the fit to various statistical distributions such as normal distributions and Rayleigh distributions. In addition, activation functions are able to apply basic arithmetic operations to several inputs instead of just adding them and applying an activation function.

As in standard feed-forward architectures, the mapping by an intermediate layer l is given by h^{l}=W^{l}o^{l}+b^{l}, where h is a vector of neuron activations for the current layer and o is a vector of neuron activations for the previous layer, computed by means of one of the functions described above. W is a matrix of weights connecting the neurons in h and o and b is a bias vector that makes an affine transformation. In the case of a single layer, the equation describes a linear hyperplane, where the entries in W control the slopes in each dimension and the entries in b shift the hyperplane up or down. The architecture is made end-to-end differentiable by means of automatic differentiation. L1 regularization may be applied to the weights to encourage sparsity and the selection of a small number of functions.

Attractor Quantification Predictor

A further predictor is an attractor quantification predictor, for example using robust attractor reconstruction. This predictor reconstructs attractor dynamics from a noisy, unevenly sampled signal and then quantifies the dynamics with various metrics. The attractor of a chaotic dynamical system may be reconstructed from a sequence of observations of one of its states, preserving the properties of the dynamical system under ideal conditions. The observed scalar time series, sampled at intervals Δt, is denoted as x(t₀+n·Δt)=x(n), where n is a counter. The reconstructed attractor is then described in d-dimensional space at time delays T by y(n)=(x(n), x(n+T), . . . , x(n+d−1)·T). The delay embedding is computed by looping over all possible values of n, from 1 until the length of the time series. For each of these values, y(n) is computed using the above equations. Each y(n) will have dimensionality d.

The reconstructed attractor can then be quantified using metrics. However, the presence of significant noise and irregular sampling can cause a breakdown in the reconstruction, and these factors characterize heart signals recorded from general purpose devices such as smartphones. If this occurs, then the reconstruction may not capture the topology of the actual attractor. Among other problems, the noise inflates the embedding dimension and can even lead to false recurrence patterns in random/stochastic systems.

To enforce topological consistency and to lower the dimensionality to avoid false recurrence, the attractor is re-embedded using multi-dimensional scaling. The reconstructed attractor is denoted as Y=(y(1), . . . , y (N)) using time delay embedding as described above. The re-embedded attractor is then obtained as Z=(z(1), z(N)) in lower dimensional space (|z(i)|<|y(i)|) such that the pairwise distances of Z are as close as possible to the pairwise distances in Y and the topology is preserved. Expressed more formally, the points in Z are found by minimizing the stress (Σ_{i,j}(∥y(i)−y(j)∥−∥zᵢ−zⱼ∥)²)/Σ_{i,j}∥y(i)−y(j)∥² as in multi-dimensional scaling. Because discrepancies between all pairwise distances are penalized, this ensures that individual outliers due to noise cannot distort the overall topology.

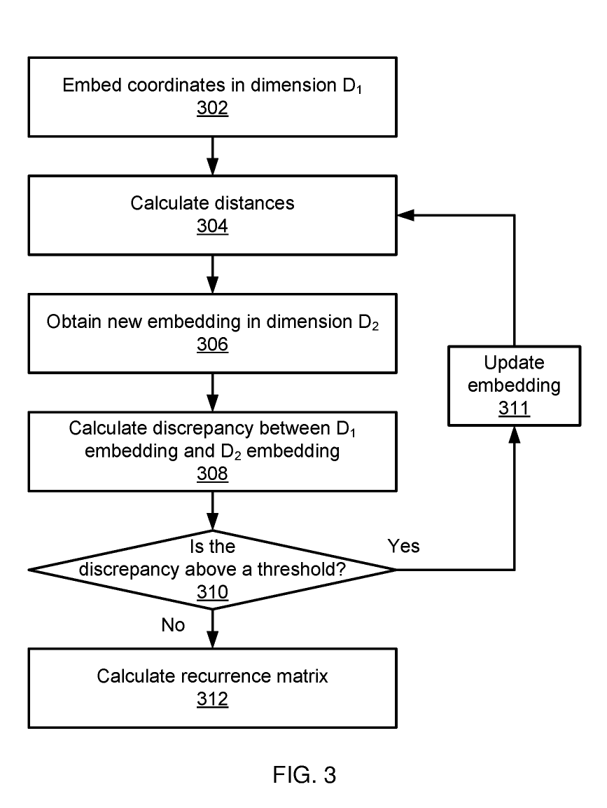

Referring now to FIG. 3, a method of reconstructing and quantifying robust attractors is shown. As noted above, block 302 embeds coordinates in a first space, D₁, having a first dimensionality d₁. This embedding is represented in the discussion above by the attractor Y. Block 304 then calculates the pairwise distances between the points in Y. Block 306 obtains a new embedding (e.g., attractor Z above) in a send space D₂ having a second dimensionality d₂.

Block 308 calculates a discrepancy between the distances in space D₁ and the distances in space D₂. If the discrepancy is above a threshold in block 310, then block 311 updates the embedding by, e.g., computing the gradient of the “stress” described above and then performing gradient descent. If not, block 312 calculates a recurrence matrix by thresholding a distance matrix at a threshold t. Based on the attractor, the recurrence matrix may be calculated as:

$${R}_{i , j} = \{ \begin{matrix}1 & if ‖ {z}_{i} - {z}_{j} ‖ < ϵ \\ 0 & otherwise\end{matrix}$$

where ∥zᵢ−zⱼ∥ represents a pairwise distance in space D₂ and ϵ represents a threshold distance. R_{i,j} can be quantified to yield additional predictors that the system may use.

Exemplary types of recurrence quantification analysis include, e.g., recurrence rate, determinism, maximum diagonal line length, entropy, and recurrence probability. Recurrence rate may be characterized as:

$$RR = 1 / {N}^{2} {\sum }_{i \cdot j = 1}^{N} {R}_{i , j}$$

where N is the number of rows (or columns in the recurrence matrix.

Determinism may be characterized as:

$$DET = \frac{{\sum }_{l = {l}_{min}}^{N} l P ( l )}{{\sum }_{i , j = 1}^{N} {R}_{i , j}}$$

where P(l) is the frequency distribution of diagonal lines of length l. Given P(l), the nominator sums up lP(l) for all possible diagonal lines of length l between a minimal length lₘᵢₙ and the maximal possible length N. The determinism is effectively the percentage of recurrence points which form diagonal lines of at least lₘᵢₙ length in the recurrence plot.

Maximum diagonal line length may be characterized as: ML=max(P(l)). Entropy may be characterized as: ENTR=−Σₗ₌ₗ_{ₘᵢₙ}_{^{N}}p(l)ln p(l). Recurrence probability may be characterized as:

$$PROB = \frac{\sum ( diag ( R , k ) )}{N - k}$$

describing the probability that the trajectory is recurrent after k time steps.

Graph-Based Predictors

An additional class of predictors can be found in graph-based methods. Predictors may be formed by a combination of a graph construction method and quantification methods. The graph construction method represents the inter-beat interval time series and the quantification methods describe the graphs. Any combination of construction and quantification methods may be used to generate a predictor.

Exemplary graph construction methods include, e.g., re-embedding graphs, mutual information graphs, and horizontal visibility graphs. A re-embedding graph is a graph constructed using the recurrence matrix R_{i,j} of the re-embedded attractor Z as its adjacency matrix, such that the adjacency matrix A=R and nodes i and j are connected if R_{i,j}=1.

A mutual information graph uses thresholded pairwise mutual information of embedding coordinates z as the adjacency matrix, such that nodes i and j are connected if MI(zᵢ, z₁)>T, where MI denotes the mutual information and T is a model parameter.

A horizontal visibility graph is a network constructed of a time series (t₁, x₁), . . . , (tₙ, xₙ), such that each x has a corresponding vertex and each pair of vertices corresponding to a pair of values xₐ and x_{b} is connected by an edge if both xₐ, x_{b}>xₙ for all a<n<b. The horizontal visibility graph is invariant under affine transformations, preserves structural properties such as periodicity and fractality, and can discriminate stochastic and chaotic processes.

Graph quantification methods are used to quantify the graphs as predictors. The graph is designated by G, lower-case characters represent vertices, V_{G} is the set of all vertices in the graph G, and d(a,b) is the distance between two vertices, defined as the shortest walk along existing graph edges. Furthermore, deg(ν) is the number of edges connected to the vertex ν and

$$ecc ( v ) = {max}_{x ∈ {V}_{G}} \{ d ( v , x ) \}$$

is the eccentricity of a vertex ν.

$$Diameter \text{:} diam ( G ) = {max}_{x ∈ {V}_{G}} \{ ecc ( x ) \} . \\ Radius \text{:} rad ( G ) = {min}_{x ∈ {V}_{G}} \{ ecc ( x ) \} .$$

Transitivity: T (G)=|Tri(G)|/|Tri(V)|, where Tri(G) is the set of all triangles in G and Tri(V) is the set of all possible triangles given all vertices V.

Cluster coefficient:

$$V ( G ) = \frac{1}{\vert {V}_{G} \vert} {\sum }_{v ∈ G} \frac{2 \vert Tri ( v ) \vert}{deg ( v ) ( deg ( v ) - 1 )} ,$$

where Tri(ν) is the set of all triangles through vertex ν.

Average shortest path length:

$$l ( G ) = {\sum }_{a , b ∈ {V}_{G}} \frac{d ( a , b )}{\vert {V}_{G} \vert ( \vert {V}_{G} \vert - 1 )} .$$

Assortativity: r(G)=(σₐσ_{b})⁻¹Σ_{xy}(e_{xy}−aₓb_{y}), where e_{xy} is the joint probability of degrees of vertices x and y and aₓ and b_{y} are the fraction of edges starting and ending at vertices x and y respectively.

Disassortative entropy: E(G)=Σ_{y}Σₓe_{xy} log e_{xy}.

Graph index complexity: C(G)=4c_r(1−cᵣ), where

$${c}_{r} = \frac{( r - 2 cos ( \frac{π}{N + 1} ) )}{N - 1 - 2 cos ( \frac{π}{N + 1} )}$$

and where r is the largest eigenvalue of the adjacency matrix of the graph.

Graph energy: Σᵢ₌₁^{N}|λᵢ|, where λᵢ are eigenvalues of the adjacency matrix.

Bertz complexity index: C(G)=2N log(N)−Σᵢ₌₁^{N}|Nᵢ|log(|Nᵢ|), where |Nᵢ| are the cardinalities of the vertex orbits (e.g., the number of vertices belonging to the respective orbits).

Edge magnitude mean information content

$$MIC = - {\sum }_{( i , j ) ∈ E} {C}_{- 1} ( G ) {( {k}_{i} {k}_{j} )}^{- \frac{1}{2}} {log}_{2} ( \frac{{( {k}_{i} {k}_{j} )}^{- \frac{1}{2}}}{C ( G )} ) ,$$

where k₁ are vertex degrees and C(G)=Σ_{(i,j)ϵE}(kᵢ,kⱼ)^{-1/2}.

where E is the set of all edges. Each (i,j)ϵE denotes a concrete edge that connects vertex I and vertex j.

Distribution Property-Based Predictors

Statistics-based predictors quantify the similarity of the time series values to two kinds of distributions: normal distributions and Rayleigh distributions. Normal distributions are relevant due to the central limit theorem, while Rayleigh distributions are relevant if the magnitude of a vector is related to several directional components, which is the case for heart signals. The time series distribution may be estimated using, e.g., kernel density estimation (KDE), and the similarity of the KDE estimate to the normal and Rayleigh distributions is quantified. Three similarity metrics are disclosed herein, though it should be understood that other similarity metrics may be used instead. The three similarity metrics yield six predictors for the system and include:

Peak separation: The distance between the maxima of the KDE distribution and the best-fitting normal/Rayleigh distribution.

KL divergence: The Kullback-Leibler divergence between the fitted KDE distribution and the best-fitting normal/Rayleigh distribution.

Area: The total absolute area between the fitted KDE distribution and the best-fitting normal/Rayleigh distribution.

In addition, some basic statistics may be used, including:

The N^{th} moment of the R-R series.

The standard deviation of the M^{th} derivative of the R-R series, where M is a model parameter that specifies which derivative to take before calculating the standard deviation.

The root mean squared error compared to the smoothed R-R series, smoothed with, e.g., a Savitzky-Golay filter with window size W.

The first zero-crossing of the generalized self-correlation function.

Discretization-Based Predictors

Discretization-based predictors look for structure and complexity in a discrete sequence instead of using a continuous time series. A first measure of complexity in the R-R time series is through the length of its compressed form. After discretizing the series by rounding each value to, e.g., three digits, the time series may be compressed using, e.g., a Lempel Ziv Welch compression process. A second discretization-based predictor builds a histogram from the R-R series and measures the Shannon entropy of the histogram. A third discretization-based predictor uses the automutual information from the same histogram as a predictor. A final discretization-based predictor discretizes the time series in four symbols, based on local derivatives: up-up, up-down, down-up, and down-down. The Shannon entropy of the discretized series is then calculated.

Forming a Model

As noted above, block 102 trains an HRV model by selecting an optimal set of predictors. Each model is trained to detect a particular disease or condition and is formed from multiple predictors and a classifier. To use one simple example, consider two time series of R-R intervals, one for a healthy person ([0.9, 0.7, 1, 0.9]) and one for a person with a particular heart condition ([0.5, 0.55, 0.5, 0.45]). For this example, the predictors may include mean inter-beat time, standard deviation of inter-beat time, and number of heart beats. For the healthy person, these predictors evaluate to [0.875, 0.109, 4] and for the person with the heart condition, these predictors evaluate to [0.5, 0.035, 4]. The classifier is trained by block 102 using a number of different examples for both healthy and unhealthy individuals and, in this case, the classifier may determine that if the mean heart beat intervals and the standard deviations are both low, then an input heart beat information indicates a heart condition.

Block 102 thereby selects the best model—the set of predictors that provide the highest degree of predictive accuracy. Following the above example, while the mean heart beat intervals and standard deviation help distinguish healthy from unhealthy, the number of heart beats does not, and so the number of heart beats might be omitted as a predictor in the best model. Thus, all of the predictors listed above may be considered, and any appropriate combination of predictors may be used to provide the most accurate model possible.

It should be understood that embodiments described herein may be entirely hardware, entirely software or including both hardware and software elements. In a preferred embodiment, the present invention is implemented in hardware and software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

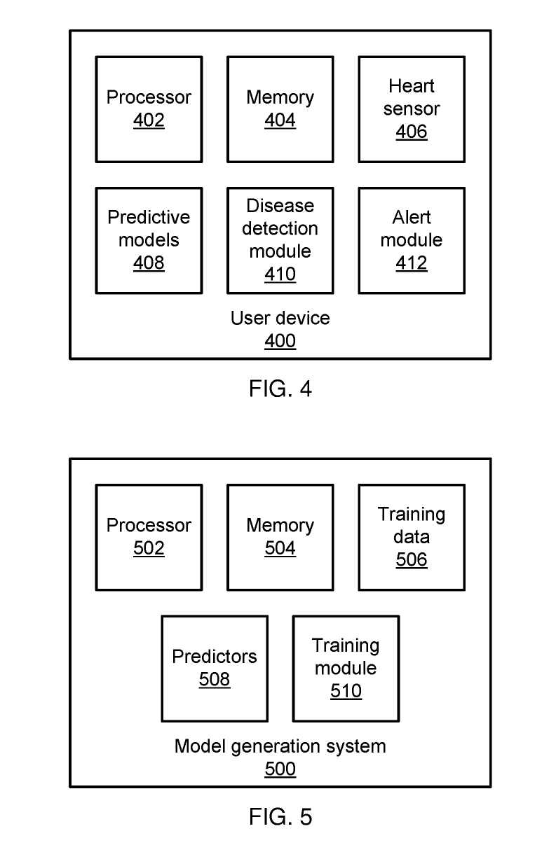

Referring now to FIG. 4, a diagram of a user device 400 is shown. While it is specifically contemplated that the user device 400 may be a user's cell phone, it should be understood that any appropriate dedicated or general-purpose device may be used instead. The user device 400 includes a hardware processor 402 and memory 404. The user device 400 also includes a heart sensor 406 which measures the user's heart beat information. The heart sensor 406 may be, for example, a dedicated heart measurement device or may, alternatively, use information collected by sensors such as a camera. In addition, the user device 400 includes one or more functional modules. In one embodiment, the functional modules may be implemented as software that is stored in the memory 404 and is executed by hardware processor 402. In an alternative embodiment, the functional modules may be implemented as one or more discrete hardware components in the form of, e.g., application-specific integrated chips or field programmable gate arrays.

For example, a disease prediction module 410 uses heart beat information captured by the heart sensor 406 and one or more predictive models 408 that are stored in memory 404 to determine whether the captured heart beat information is indicative of a disease or other heart condition. As noted above, the predictive models 408 include a classifier and a set of predictors that can be used to recognize such conditions. If a disease or other heart condition is detected, an alert module 412 provides an audio or visual alert to the user or to a health care provider. The alert may be a simple warning or indicator or may, alternatively, provide detailed information about the detected condition.

Referring now to FIG. 5, a model generation system 500 is shown. It is specifically contemplated that model generation and training may be performed offline, with models being subsequently distributed to user devices 400. The model generation system 500 includes a hardware processor 502 and memory 504. In addition, model generation system 500 includes one or more functional modules. In one embodiment, the functional modules may be implemented as software that is stored in the memory 504 and is executed by hardware processor 502. In an alternative embodiment, the functional modules may be implemented as one or more discrete hardware components in the form of, e.g., application-specific integrated chips or field programmable gate arrays.

In particular, a training module 510 accesses a corpus of training data 506 that is stored in memory 504, where the training data 506 includes sets of stored heart beat information for known-healthy and known-unhealthy individuals. The training module 510 accesses a set of uncorrelated predictors 508, each of which can be used to predict the presence of a disease or other heart condition based on heart beat information. The training module 510 finds an optimal set of predictors 508 to include in a model and trains a classifier based on those predictors 508 that most effectively discriminates between healthy and unhealthy individuals. The training module 510 may generate multiple such models for respective diseases or heart conditions. The generated models can then be provided to user devices 400 for use in detecting such diseases in the field.

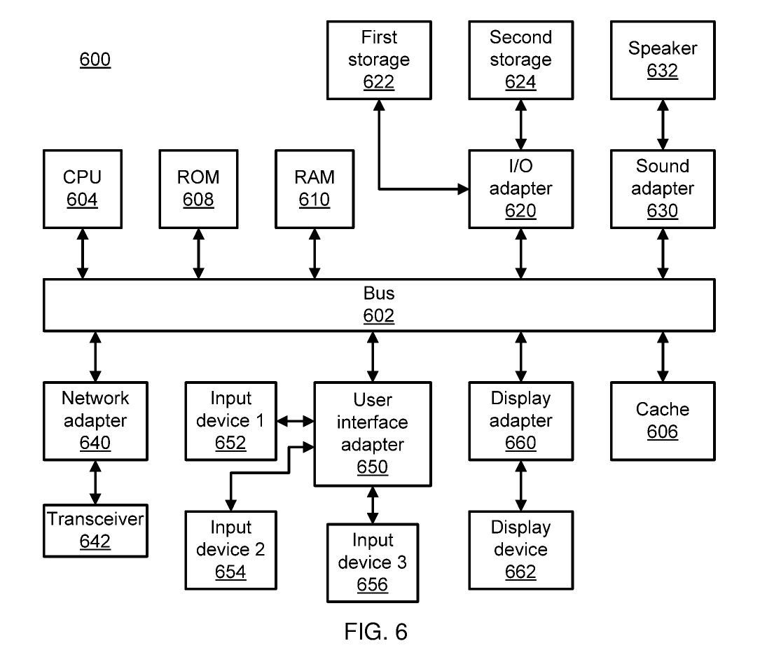

Referring now to FIG. 6, an exemplary processing system 600 is shown which may represent the user device 400 or the model generation system 500. The processing system 600 includes at least one processor (CPU) 604 operatively coupled to other components via a system bus 602. A cache 606, a Read Only Memory (ROM) 608, a Random Access Memory (RAM) 610, an input/output (I/O) adapter 620, a sound adapter 630, a network adapter 640, a user interface adapter 650, and a display adapter 660, are operatively coupled to the system bus 602.

A first storage device 622 and a second storage device 624 are operatively coupled to system bus 602 by the I/O adapter 620. The storage devices 622 and 624 can be any of a disk storage device (e.g., a magnetic or optical disk storage device), a solid state magnetic device, and so forth. The storage devices 622 and 624 can be the same type of storage device or different types of storage devices.

A speaker 632 is operatively coupled to system bus 602 by the sound adapter 630. A transceiver 642 is operatively coupled to system bus 602 by network adapter 640. A display device 662 is operatively coupled to system bus 602 by display adapter 660.

A first user input device 652, a second user input device 654, and a third user input device 656 are operatively coupled to system bus 602 by user interface adapter 650. The user input devices 652, 654, and 656 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input devices 652, 654, and 656 can be the same type of user input device or different types of user input devices. The user input devices 652, 654, and 656 are used to input and output information to and from system 600.

Of course, the processing system 600 may also include other elements (not shown), as readily contemplated by one of skill in the art, as well as omit certain elements. For example, various other input devices and/or output devices can be included in processing system 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 600 are readily contemplated by one of ordinary skill in the art given the teachings of the present principles provided herein.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. Additional information is provided in Appendix A to the application. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention.

1. A method of detecting a heart condition, comprising:
measuring a user's heart beat information;
applying a predictive model, using a processor, that comprises a plurality of individual predictors and a classifier, wherein each predictor of the plurality of individual predictors maps the user's heart beat information to a respective value, said classifier indicating a likelihood of a heart condition based on the plurality of individual predictors and the user's heart beat information; and
issuing an alert if the likelihood is above a threshold value.

2. The method of claim 1, wherein the plurality of predictors comprises a biological neuron model based predictor.

3. The method of claim 2, wherein the biological neuron model based predictor comprises a neural network comprising one or more hidden neuron layers and an input neuron layer, wherein neurons in the input neuron layer represent non-linear dynamical systems and provide firing rates to a first hidden neuron layer.

4. The method of claim 1, wherein the plurality of predictors comprises a neuronal equation learning predictor.

5. The method of claim 1, wherein the plurality of predictors comprises a robust attractor reconstruction predictor comprising a recurrence matrix.

6. The method of claim 5, wherein applying the predictive model comprises:
embedding the heart beat information in a first space having a first dimensionality;
embedding the heart beat information in a second space having a second dimensionality;
iteratively updating the embedding in the second space until a discrepancy between the embeddings falls below a threshold value.

7. The method of claim 5, wherein the robust attractor reconstruction predictor further comprises a metric that quantifies the recurrence matrix.

8. The method of claim 1, wherein the plurality of predictors comprises a graph-based predictor.

9. The method of claim 8, wherein the graph-based predictor generates a graph selected from the group consisting of a re-embedding graph and a mutual information graph.

10. The method of claim 8, wherein the graph-based predictor quantifies a graph based on a dissassortative entropy of the graph.

11. A system for detecting a heart condition, comprising:
a sensor configured to measure a user's heart beat information;
a disease detection module comprising a processor configured to apply a predictive model that comprises a plurality of individual predictors and a classifier, wherein each predictor of the plurality of individual predictors maps the user's heart beat information to a respective value, said classifier indicating a likelihood of a heart condition based on the plurality of individual predictors and the user's heart beat information; and
an alert module configured to issue an alert if the likelihood is above a threshold value.

12. The system of claim 11, wherein the plurality of predictors comprises a biological neuron model based predictor.

13. The system of claim 12, wherein the biological neuron model based predictor comprises a neural network comprising one or more hidden neuron layers and an input neuron layer, wherein neurons in the input neuron layer represent non-linear dynamical systems and provide firing rates to a first hidden neuron layer.

14. The system of claim 11, wherein the plurality of predictors comprises a neuronal equation learning predictor.

15. The system of claim 11, wherein the plurality of predictors comprises a robust attractor reconstruction predictor comprising a recurrence matrix.

16. The system of claim 15, wherein the disease detection module is further configured to embed the heart beat information in a first space having a first dimensionality, to embed the heart beat information in a second space having a second dimensionality, and to iteratively update the embedding in the second space until a discrepancy between the embeddings falls below a threshold value.

17. The system of claim 15, wherein the robust attractor reconstruction predictor further comprises a metric that quantifies the recurrence matrix.

18. The system of claim 11, wherein the plurality of predictors comprises a graph-based predictor.

19. The system of claim 18, wherein the graph-based predictor generates a graph selected from the group consisting of a re-embedding graph and a mutual information graph.

20. The system of claim 18, wherein the graph-based predictor quantifies a graph based on a dissassortative entropy of the graph.
